(19) United States
(12) Patent Application Publication
(10) Pub. No.: US 2019/0117331 A1
(43) Pub. Date: Apr. 25, 2019

(54) SURGICAL DRAPES FOR REDUCING SURGICAL SITE INFECTIONS

(71) Applicants: Sean D. Self, Sugar Land, TX (US); Geoff A. Marcek, Austin, TX (US); James M. Fowler, JR., Houston, TX (US)

(72) Inventors: Sean D. Self, Sugar Land, TX (US); Geoff A. Marcek, Austin, TX (US); James M. Fowler, JR., Houston, TX (US)

(21) Appl. No.: 16/142,267

(22) Filed: Sep. 26, 2018

Related U.S. Application Data
(60) Provisional application No. 62/566,888, filed on Oct. 2, 2017.

(51) Int. Cl. A61B 90/40; A61B 46/20

(52) U.S. Cl. CPC A61B 90/40; A61B 2090/401; A61B 2046/205; A61B 46/20

(57) ABSTRACT

A surgical drape for preventing surgical site infections during surgery includes an emitter sealed air tight to the drape. The emitter includes an inflatable receiver portion and a terminal outlet distributor fluidly continuous with the receiver portion. The receiver portion inflates on receiving a sterile gas flow from a source of sterile gas. The terminal outlet portion conditions the received sterile gas and emits it as a unidirectional coherent non-turbulent flow field of essentially sterile gas into ambient air in the operating room substantially anatomically levelly at least immediately past the incision while keeping the gas essentially sterile. An embodiment is for use in surgery on an extremity of the patient and another embodiment is for use in surgery on an anatomical part that is not an extremity.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE DISCLOSURE

### Field of the Disclosure

This invention relates to surgery, and more particularly, to medical drapes used to reduce contamination of surgical sites.

### Background Art

Hospital-acquired infections (“HAI”), also known as nosocomial infections, are a significant problem in modern healthcare systems. In 2010, an estimated 16 million operative procedures were performed in acute care hospitals in the United States. Center for Disease Control and Prevention (“CDC”), Data from the National Hospital Discharge Survey, 2010, available at: www.cdc.gov/nchs/data/nhds/4procedures/2010pro_numberpercentage .pdf. A recent prevalence study found surgical site infections (“SSIs”) were the most common healthcare-associated infection, accounting for 31% of all HAIs among hospitalized patients. Magill, S. S., et al., Prevalence of healthcare-associated infections in acute care hospitals in Jacksonville, Fla., INFECTION CONTROL HOSPITAL EPIDEMIOLOGY, 33(3):(2012): 283-91. A CDC HAI prevalence survey found that in 2011 there were an estimated 157,500 surgical site infections associated with inpatient surgeries. Magill, S. S., et al., Multistate point-prevalence survey of health care-associated infections. NEW ENGLAND JOURNAL OF MEDICINE, 370(13): (2014): 1198-1208.

SSI infections mostly are “staph” infections, frequently caused by the bacterium Staphylococcus aureus and/or Staphococcus epidermidis, which occur harmlessly on human skin and frequently in the nose. If these bacteria gain access though an incision slice to a normally sterile space they may multiply without resistance and create a huge infectious burden on the host. These types of infections develop deep within the body, are difficult to treat, and are devastating to patients. SSI from bacterial invasion is particularly deleterious in procedures such as orthopedic joint arthroplasty, cardiovascular surgery, and neurosurgery. As respects orthopedic joint arthroplasty, the total number of implant infections that occur after total hip and knee arthroplasty in the United States is projected to increase from 22,000 in 2009 to 65,000 in 2020, with healthcare costs exceeding $1 billion per year by 2020. Kurtz S M, Lau E, Watson H, Schmier J K, Parvizi J., Economic burden of periprosthetic joint infection in the United States. J ARTHROPLASTY 2012; 27:61-65.

SSI is associated with a mortality rate of 3%, and 75% of SSI-associated deaths are directly attributable to the SSI. CDC National and State Healthcare-Associated Infections Progress Report, published March 2016, available at: www.cdc.gov/hai/surveillance/progress-report/index.html

While advances have been made in infection control practices, including improved operating room ventilation, sterilization methods, barriers, surgical technique, and availability of antimicrobial prophylaxis, SSIs remain a substantial cause of morbidity, prolonged hospitalization, and death.

Bacteria are generally 0.5-1 μm in size or larger and have a tendency to cluster together and attach to other larger particles. Airborne bacteria-carrying particles measure about 4 μm to 20 μm. Humans constantly shed skin scales in the 5-20 μm particle range into the atmosphere. As reported in Stocks G W, Self S D, Thompson B, Adame X A, O'Connor D P, Predicting bacterial populations based on airborne particulates: a study performed in nonlaminar flow operating rooms during joint arthroplasty surgery. AM J INFECT CONTROL 2010; 38:199-204, the number of airborne particles 10 μm or larger in size present in an operating room during actual hip and knee joint arthroplasties conducted under carefully controlled testing, but without benefit of the invention described herein, was associated with the number of bacterial colony forming units (CFU) grown from air sampled within the sterile field approximately 40 cm from the surgical incision at the surgical site during the arthroplasties. (Reference herein to 10 μm particles is to be understood to mean 10 μm and larger.) The number of 10 μm airborne particles was also associated with the number of surgical staff present in the operating room. The number of people, door openings, and room traffic all increase the quantity of airborne colony-forming units (CFU). The finding that the number of 10 μm particles was correlated with the number of CFU at the surgical site supports airborne particulate contamination of the wound as a source of post-operative infection in joint arthroplasty. When the density of 10 μm particles in these operating rooms exceeded 300 particles/ft³ in any 10-min interval, the average CFU count at the surgical site exceeded 25 CFU/m³ during that interval. It is likely that the correlation of larger particles (10 μm and larger) with CFUs observed in this study was attributable to the larger particles being capable of carrying bacteria. U.S. Pat. Nos. 7,409,129 and 9,191,727 describe our device and methodology for preventing particles 5 μm and larger from entering an incision slice to a normally sterile space during surgery. The efficacy of the technology was recently described in Darouche, R. O.; Green, D. M.; Harignton, M. A.; Ehni, B. L.; Kougias, P.; Bechara, C. B.; and O'Connor, D. P., Association of Airborne Microorganisms in the Operating Room with Implant Infections: A Randomized Controlled Trial, INFECTION CONTROL AND HOSPITAL EPIDEMIOLOGY, 2016; 1-8.

Various types of surgical drapes have been used to keep a surgical site on a patient sterile during a surgical procedure. In surgical procedures, the site of an incision is typically prepared first by painting the surgical area with an iodine or other bactericidal solution, followed by laying over the area a film the underside of which is coated with an adhesive and bactericide, and then by placing over the film a disposable drape having a window (a “fenestration”) for the incision work and next by adhering the drape to the film by an adhesive on the underside borders of the fenestration. Once prepared, an incision is made within the window through the film into the painted skin. These drapes are not designed to prevent airborne particles 5 μm and larger from entering an incision slice during surgery and are not effective for that purpose.

There is a need to bring this technology to the field of sterile surgical drapes to order to exclude airborne particles 5 μm and larger from the surgical field and help protect the patient from the growing scourge of SSI and reduce the exploding costs of healthcare.

### BRIEF DESCRIPTION OF THE DRAWINGS

FIG. 1. is a perspective view of an embodiment of the topside of a surgical drape including an embodiment of a sterile gas conduit joined to the topside and a flow conditioning sterile gas emitter sealed to the topside of the drape.

FIG. 2 is a perspective view of the bottom side of the surgical drape embodiments of FIG. 1.

FIG. 3 is a perspective view of the sterile gas emitter of FIG. 1 with distributor pictured in FIG. 4 removed.

FIG. 4 is a frontal and top plan view of a frontally planar foam air distributor employed in the sterile gas emitter of FIG. 3.

FIG. 5 is a perspective view of a topside of a surgical drape.

FIG. 6 is a perspective view of an embodiment of the topside of a surgical drape of FIG. 5 modified to include an embodiment of a sterile gas conduit penetrating the drape from a bottom side location and a flow conditioning sterile gas emitter connected to the sterile gas conduit and sealed to the topside of the drape.

FIG. 7 is a perspective view of the sterile gas emitter of FIG. 6.

FIG. 8 is a side and end view of a sterile gas conduit of FIG. 6.

FIG. 9 is a perspective view of a sterile gas emitter as it would appear inflated, but with the frontally planar foam air distributor removed.

FIG. 10 is a perspective view of a frontally planar foam air distributor for fitment in the sterile gas emitter of FIG. 9.

FIG. 11 is a perspective view of a bezel of an anterior terminal portion of the emitter of FIG. 11 to which an inflatable receiver portion of the emitter of FIG. 13 is attached.

FIG. 12 is a perspective view of a ring to which the inflatable receiver portion is posteriorly attached and to which a sterile gas conduit connects.

FIG. 13 is an assembly of the components of FIGS. 9-12.

FIG. 14 is a perspective view of an embodiment of the topside of a surgical drape including an embodiment of a sterile gas conduit and a deflated flow conditioning sterile gas emitter coupled to the topside of the drape in accordance with the invention.

FIG. 15 is an enlarged perspective view of the deflated sterile gas emitter of FIG. 14.

FIG. 16 is a perspective view of an embodiment of a surgical drape in which a terminal outlet portion is an annulus and a distributor within said annulus is annular for surroundingly accepting an extremity of a patient though said distributor with a portion of the extremity where an incision is to be made extending through the distributor.

FIG. 17 is an enlargement of the area “A” shown in FIG. 16.

FIG. 18 is a diagrammatic sketch of a longitudinal section of the FIG. 17 enlargement showing a sleeve into which an extremity of the patient is inserted.

### DETAILED DESCRIPTION OF EMBODIMENTS

In the following detailed description of embodiments, reference is made to the accompanying drawings, which form a part hereof and in which are shown, by way of illustration, specific embodiments in which the invention may be practiced. Specific details disclosed herein are in every case a non-limiting embodiment representing concrete ways in which the concepts of the invention may be practiced. This serves to teach one skilled in the art to employ the present invention in virtually any appropriately detailed system, structure or manner consistent with those concepts. It will be seen that various changes and alternatives to the specific described embodiments and the details of those embodiments may be made within the scope of the invention. Because many varying and different embodiments may be made within the scope of the inventive concepts herein described and in the specific embodiments herein detailed without departing from the scope of the present invention, it is to be understood that the details herein are to be interpreted as illustrative and not as limiting.

The various directions such as “upper,” “lower,” “bottom,” “top,” “back,” “front,” “perpendicular”, “vertical”, “horizontal,” “length” and width” and so forth used in the detailed description of embodiments are made only for easier explanation in conjunction with the drawings to express the concepts of the invention. The elements in embodiments may be oriented differently while performing the same function and accomplishing the same result as obtained with the embodiments herein detailed, and such terminologies are not to be understood as limiting the concepts which the embodiments exemplify.

As used herein, the use of the word “a” or “an” when used in conjunction with the term “comprising” (or the synonymous “having” or “including”) in the claims and/or the specification may mean “one,” but it is also consistent with the meaning of “one or more,” “at least one,” and “one or more than one.” In addition, as used herein, the phrase “connected to” means joined to or placed into communication with, either directly or through intermediate components.

In accordance with the present invention, the embodiments of surgical drapes described herein work to create a figurative “cocoon” of essentially sterile gas immediately overlaying a surgical opening, sometimes called a surgical wound, to protect the wound from contamination by ambient airborne particles. The cocoon is a localized flow field of coherent non-turbulent essentially sterile gas. The word “gas” is used to include mixtures, compounds and elemental gases that are not deleterious for surgical use, and includes air, mixtures of air with vapors or other gases having a sterilant property, i.e., having active properties that help negate viability of microbes, such as hydrogen peroxide or ozone, mixtures of air with vapors that have analgesic properties, and mixtures of air with low concentrations of one or more denser inert gases, such as nitrogen or argon, to displace ambient air. Air is mostly used as a source gas for scrubbing by a HEPA (“high efficiency particulate air”) filter device to provide a sterile gas because it is free of added cost. The term “sterile gas” means gas from which 90% or more (suitably and preferably 99.997%) of ambient airborne particulates 0.3 μm and larger have been removed. The term “essentially sterile gas” is used to mean a gas containing 90% or less of particles 5 μm and larger than in ambient air in the room where the embodiments are employed.

The “cocoon” of essentially sterile gas is created to be located beneath both surgical staff (who are usually leaning over the incision) and the overhanging operating room equipment, with the result that surgical staff, operating room equipment and others in the operating room, who are a source of airborne particles that may carry microbes, are positioned outside the protective cocoon. As used herein, “microbes” includes bacteria, fungal spores and other microorganisms present in the ambient atmosphere in an operating room.

In accordance with the present invention, the cocooned protection of the surgical site is achieved by attaching a unidirectional coherent non-turbulent flow field of essentially sterile gas substantially anatomically levelly on an anatomical surface of a patient adjacent the site of a surgical incision, and maintaining that flow field substantially anatomically levelly along the surface anatomy of the patient up to and through the incision site while keeping the gas essentially sterile. The flow field sweeps ambient airborne particles along the outer layers of the flow field cocoon away from the interior layers over the surgical wound. The supply of sterile gas preferably is conditioned into a unidirectional coherent non-turbulent flow field having a velocity in the range from about 180 to 400 ft/min and flowed at a rate of from about 10 ft3/min to 50 ft3/min in a flow field extending a distance in the range of from about 3 inches to about 20 inches. The flow field transitions to turbulent flow past the field of effect disbursing ambient particles away from the operating table.

In order to accomplish this, there is provided a surgical drape for affixation onto an anatomical surface adjacent a site of incision typical for a type of surgery to protect a patient from surgical site infection during the surgery. The surgical drape comprises anatomically shape conforming means for attaching a unidirectional coherent non-turbulent flow field of sterile gas substantially anatomically levelly on said anatomical surface flowing in the direction of the incision site and preventing ambient airborne particles from entering the interior of the flow field under said means sufficiently to maintain said gas essentially sterile at the incision site. Embodiments of surgical drapes disclosed herein provide the means for accomplishing the stated protection.

In an embodiment of this invention, a surgical drape comprises a topside, a bottom side, and an emitter sealed to a portion of the drape. The emitter comprises an inflatable receiver portion for receipt of a flow of sterile gas and a terminal outlet portion fluidly continuous with the receiver portion. The terminal outlet portion contains a frontally planar gas distributor. The receiver portion pneumatically inflates upon receipt of a sterile gas flow producing backpressure at the distributor. The distributor of terminal outlet portion upon inflation of the emitter conditions the received sterile gas flow into a coherent non-turbulent flow emitted unidirectionally from the terminal outlet portion.

In an embodiment of the surgical drape the emitter has a bottom side sealed to the topside of the drape. In another embodiment of the surgical drape, at least a portion of the topside is pneumatically nonporous, the emitter has lateral sides and no bottom side, and the emitter is sealed at the edges of the lateral sides to the pneumatically nonporous portion of the topside.

In an embodiment of the surgical drape, a gas conduit is joined to the drape for receipt of sterile gas flow from a source of sterile gas and connection to the receiver portion of the emitter. In one embodiment, the gas conduit has a bottom or top side bonded respectively to the topside or bottom side of the drape, and is inflatable on receipt of sterile gas flow from a source of sterile gas. In another embodiment, at least a portion of the topside extending from the emitter distally to an inlet for connection to a source of sterile gas is pneumatically nonporous, the gas conduit has lateral sides and no bottom side and is sealed at the edges of the lateral sides to the pneumatically nonporous portion of the topside. In another embodiment of the surgical drape, the drape is combined with a gas conduit comprising a hose not joined to the drape for connection to the receiver portion of the emitter on the topside of the drape.

In an embodiment of the surgical drape, the distributor fills the terminal outlet portion of the emitter. In one such embodiment, the terminal outlet material is a different material than an inflatable material of said receiver portion and a forepart of the material of said receiver portion is sealed to the outside of the terminal outlet portion. In another such embodiment, the terminal outlet portion is a continuation of material the same as the material of which the receiver portion is formed and in which the continuation material is sealingly bonded to the perimeter of the distributor.

In an embodiment of the surgical drape in which the distributor fills the terminal outlet portion of the emitter, the surgical drape includes an opening extending completely through the drape, the opening being bounded by bordering sides, and the bottom side has an adhesive surrounding the opening for releasable adherence of the drape about a site of the surface anatomy of a patient where a surgical incision is to be made. The adhesive may be covered by a removable strip for removable of the strip and releasable adherence of the drape about such site. A surgical drape in which the distributor fills the terminal outlet portion of the emitter and in which the drape has an opening as described, the opening is for attachment to a portion of a patient that is not an extremity and the flow emitted from the terminal outlet portion proceeds levelly over the opening from only one bordering side of the opening. In one such embodiment, the opening is quadrilateral. In an embodiment, the terminal outlet portion of the emitter is close to one of the sides of the opening. By “close to” is meant a range of closeness from “next to” the bordering opening side to “near to” (up to 4 inches) from the bordering side of the opening. In one such embodiment, the terminal outlet portion of the emitter is frontally as wide as a bordering side of the opening to which the terminal outlet portion of the emitter is close.

In an embodiment of the surgical drape in which the distributor fills the terminal outlet portion of the emitter and in which the drape has an opening as described, at least a portion of the topside is pneumatically nonporous, the emitter has lateral sides and no bottom side, the emitter is sealed at the edges of the lateral sides to the pneumatically nonporous portion of the topside, and the pneumatically nonporous portion of the topside is close to one of the boundary sides of the opening.

In another embodiment of the surgical drape, the drape is applicable to surgery of an extremity of a patient. The terminal outlet portion is an annulus and the distributor within the annulus is annular and externally bonded to an interior of the annulus for surroundingly accepting an extremity of a patient though the distributor. In such an embodiment, an embodiment includes a sleeve externally bonded to an interior of the inflatable receiver portion of the emitter distal from the annulus and the sleeve is externally bonded nonporously to an interior circumference of the annular distributor, the sleeve accepting the extremity with a portion of the extremity extending past the distributor. In an embodiment, the sleeve ends in a cuff immediately past the annulus and the cuff has an interior that includes an adhesive covered by a removable strip. In such an embodiment, the flow emitted from the emitter outlet portion proceeds to a site of incision coherently generally perpendicular to a radius of the extremity.

In an embodiment of the surgical drape, the drape is foldable essentially flat with the emitter collapsed within the drape, suitably within folds of the drape. In an embodiment in which the gas conduit is also inflatable, the drape is foldable essentially flat with the gas conduit collapsed within the drape, suitably within folds of the drape.

In an embodiment of the surgical drape the receiver portion of the emitter comprises a roof of larger inflated area than an area of the topside of the drape under the roof. In an embodiment, the emitter is formed of a flexible plastic. In an embodiment the emitter is formed of a flexible plastic film.

An embodiment of the invention includes a surgical procedure kit comprising a folded essentially flat surgical drape containing the drape a deflated inflatable gas conduit joined to the drape and an emitter, the drape having a topside and a bottom side, at least a portion of the topside being pneumatically nonporous, the emitter being hermetically sealed to at least a portion of the pneumatically nonporous topside of the drape, the emitter comprising a deflated inflatable receiver portion having an inlet sealingly connected to the gas conduit and a terminal outlet portion fluidly continuous with the receiver portion, the terminal outlet portion containing a frontally planar gas distributor, the receiver portion pneumatically inflating upon receipt of sterile gas flow from the gas conduit producing backpressure at the distributor, the distributor of the terminal portion outlet upon inflation of the emitter conditioning the received sterile gas flow into a coherent non-turbulent flow emitted unidirectionally from the outlet.

In an embodiment of the invention comprising a surgical procedure kit for a patient having a surgery site that is not an extremity, the distributor fills the terminal outlet portion of the emitter and the terminal outlet portion is prone when said receiver portion is deflated, the drape further comprising an opening extending completely through the drape, the opening being bounded by bordering sides, the bottom side having an adhesive surrounding the opening for releasable adherence of the drape about a site of the surface anatomy of a patient that is not an extremity where a surgical incision is to be made, the pneumatically nonporous portion of the topside being close to one of the bordering sides of the opening. In one such embodiment, the opening is quadrilateral, the terminal outlet portion of the emitter is frontally as wide as a bordering side of the opening where the portion of the drape topside is pneumatically nonporous, and on admission of gas into the gas conduit inflating the receiving portion of the emitter, the pneumatic inflation of the receiver portion erects the terminal outlet portion, and the conditioned flow emitted from the terminal outlet portion proceeds levelly over the opening from only one bordering side of the opening.

In another embodiment comprising a surgical procedure kit for a patient having a surgery site that is an extremity, the terminal outlet portion is an annulus and the distributor within the annulus is annular and externally bonded to an interior of the annulus, the drape includes a sleeve externally bonded to an interior of the inflatable receiver portion distal from the annulus and is externally bonded nonporously to an interior circumference of the annular distributor, the sleeve accepting the extremity with a portion of the extremity extending past the distributor, and the sleeve ending in a cuff immediately past the annulus, the cuff having an interior that includes an adhesive covered by a removable strip.

A method of using a surgical procedure kit to protect a patient from surgical site infection from an incision in the anatomy of the patient that is not an extremity during surgery in an operating room where the site is exposed to operating room atmosphere, comprises (a) providing a surgical procedure kit comprising a folded essentially flat surgical drape having a topside, a bottom side, and an opening extending completely through the drape, the opening being bounded by bordering sides, the bottom side having an adhesive surrounding the opening, the drape containing within the drape a deflated inflatable gas conduit coupled to the drape and an emitter, at least a portion of the topside close to one of the sides of the opening being pneumatically nonporous, the emitter being hermetically sealed to at least a portion of the pneumatically nonporous topside of the drape, the emitter comprising a deflated inflatable receiver portion having an inlet sealingly connected to the gas conduit and a prone terminal outlet portion fluidly continuous with the receiver portion, the terminal outlet portion containing a frontally planar gas distributor filling the terminal outlet portion, the gas conduit pneumatically inflating upon receipt of sterile gas flow from a source of sterile gas and the receiver portion pneumatically inflating upon receipt of sterile gas flow from the gas conduit producing backpressure at the distributor and erecting said terminal outlet portion, the distributor of the terminal outlet portion upon inflation of the emitter conditioning the received sterile gas flow into an emitted coherent non-turbulent flow, (b) unfolding the drape and arranging the drape over the patient with the opening placed over a site of the surface anatomy of a patient that is not an extremity where a surgical incision is to be made, such placement locating the flow conditioning terminal outlet portion of the emitter at one side of the site of the surgical incision, (c) adhering the drape to the patient at the sides of the opening thereby anatomically shape conforming the flow conditioning terminal outlet portion of the emitter to the anatomical surface of a patient, (d) connecting the gas conduit to a source of sterile gas, and (e) supplying a flow of sterile gas to the gas conduit and the emitter to pneumatically inflate the gas conduit and the receiver portion of the emitter and erect the terminal outlet portion to emit a conditioned unidirectional coherent non-turbulent flow field of essentially sterile gas into ambient air in the operating room substantially anatomically levelly over the anatomical surface of the patient within the opening from one side of the site of incision to an opposite side thereof for a distance at least immediately past the incision while keeping the gas essentially sterile, the sealing of the emitter inclusive of the distributor to the pneumatically nonporous topside of the drape and the adherence of the drape to the patient preventing entry of ambient air under the emitter into the emitted flow.

A method of using a surgical procedure kit to protect a patient from surgical site infection from an incision in the anatomy of the patient that is an extremity during surgery in an operating room where the site is exposed to operating room atmosphere, comprises (a) providing a surgical procedure kit comprising a folded essentially flat surgical drape having a topside and a bottom side, at least a portion of the topside being pneumatically nonporous, the drape containing within the drape a deflated inflatable gas conduit coupled to the drape and an emitter, the emitter being hermetically sealed to at least a portion of the pneumatically nonporous topside of the drape, the emitter comprising a deflated inflatable receiver portion having an inlet sealingly connected to the gas conduit and a terminal outlet portion in the form of an annulus fluidly continuous with the receiver portion, the terminal outlet portion annulus containing an annular frontally planar gas distributor externally bonded to an interior of the annulus, the drape including a sleeve externally bonded to an interior of the inflatable receiver portion of the emitter distal from the terminal outlet portion annulus and externally bonded nonporously to an interior circumference of the annular distributor, the sleeve ending in a cuff immediately past the terminal outlet portion annulus, the cuff having an interior that includes an adhesive covered by a removable strip, the sleeve accepting an extremity of a patient with a portion of the extremity extending through and past the annular distributor, the gas conduit pneumatically inflating upon receipt of sterile gas flow from a source of sterile air, the receiver portion pneumatically inflating upon receipt of sterile gas flow from the gas conduit producing backpressure at the annular distributor, and the annular distributor upon inflation of the terminal outlet portion annulus conditioning the received sterile gas flow into an emitted coherent non-turbulent flow from the terminal annulus portion outlet, (b) unfolding the drape and inserting the extremity of the patient into the sleeve with a portion of the extremity where an incision is to be made extending through the distributor past the cuff, such placement locating the flow conditioning terminal outlet portion annulus at one side of the site of the surgical incision, (c) removing the strip from the adhesive and adhering the cuff to the extremity at the cuff thereby anatomically shape conforming the flow conditioning terminal outlet portion annulus to the anatomical surface of a patient where an incision is to be made, (d) connecting the gas conduit to a source of sterile gas flow, and (e) supplying a flow of sterile gas to the gas conduit and the emitter to pneumatically inflate gas conduit and receiver portion of the emitter to emit a conditioned unidirectional coherent non-turbulent flow field of essentially sterile gas into ambient air in the operating room substantially anatomically levelly generally perpendicular to a radius of the extremity a distance at least immediately past the incision while keeping the gas essentially sterile, the sealing of the emitter inclusive of the distributor to the pneumatically nonporous topside of the drape and the adherence of the drape cuff to the patient preventing entry of ambient air under the emitter into the emitted flow.

The surgical drapes, surgical procedure kits and methods of use of the described embodiments have numerous advantages in addition to protecting a patient from surgical site infection of an incision during surgery in an operating room where the site is exposed to operating room atmosphere. The devices of he embodiments cover the entire area of the surgical site access whereas the devices of U.S. Pat. Nos. 7,409,129 and 9,191,727 are premolded and have a fixed outlet area that may or may not match the width of the particular surgical site access. Relatedly there is less potential for error in applying the emitter embodiments to a patient than in the devices of U.S. Pat. Nos. 7,409,129 and 9,191,727, because the instant embodiments are self-aiming when the drape is applied. Apart from the sterility of the gas flow over the patient's surgical site, sterility in the operating theater is increased because there is no contact with a separate device as in the devices of U.S. Pat. Nos. 7,409,129 and 9,191,727. The devices of U.S. Pat. Nos. 7,409,129 and 9,191,727 as currently commercially embodied use two separate means of securing the emitter to a patient; they use a Velcro® pad portion applied on top of a drape and then a second mating piece of Velcro® on the bottom of the rigid emitter to adhere the emitter to a patient. The two separate means of securing the emitter to the patient are avoided in the present embodiments.

Additionally, the surgical drape of the instant embodiments by employing a collapsible pneumatically inflatable emitter as part of the drape allows more freedom of movement of a surgeon; for example, when using long lever bone retractors, the surgeon would not encounter a hard emitter surface that would actively block the intended instrument manipulation. Further, compared to the devices of U.S. Pat. Nos. 7,409,129 and 9,191,727, no separate packaging of drape and device is involved so the instant drape of the embodiments takes up less space on instrument tables in the operating theater and less storage space in hospital supplies inventory. This also means that the drape of the instant embodiments reduces the count of surgical items that have to be accounted for after surgery.

These are all advantages that have the potential to widen the solution initiated by the devices of U.S. Pat. Nos. 7,409,129 and 9,191,727 and reduce the growing scourge of SSI and resultant explosion of health care costs.

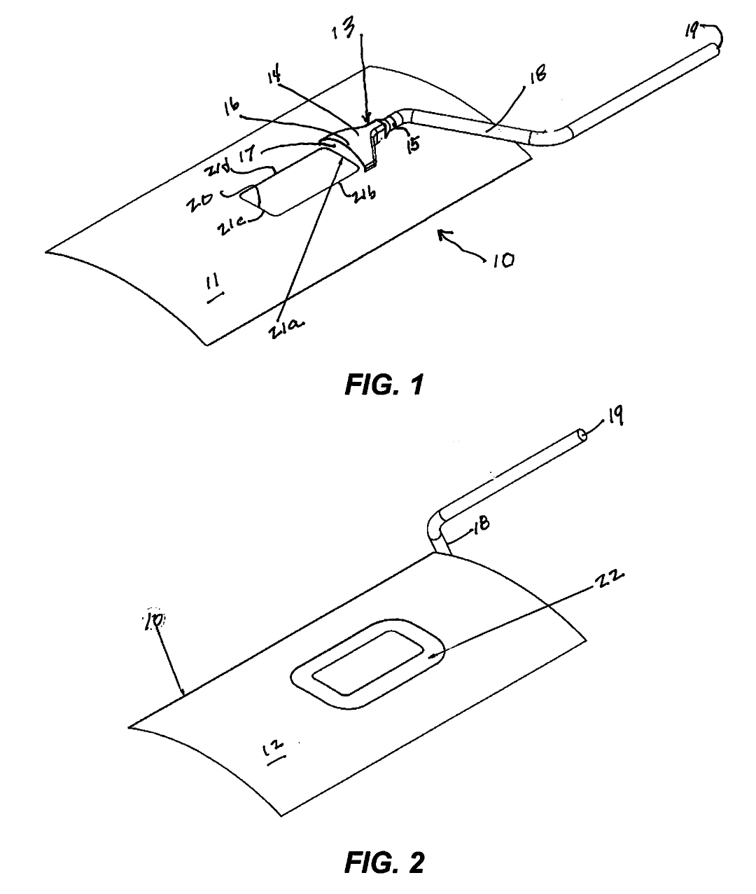

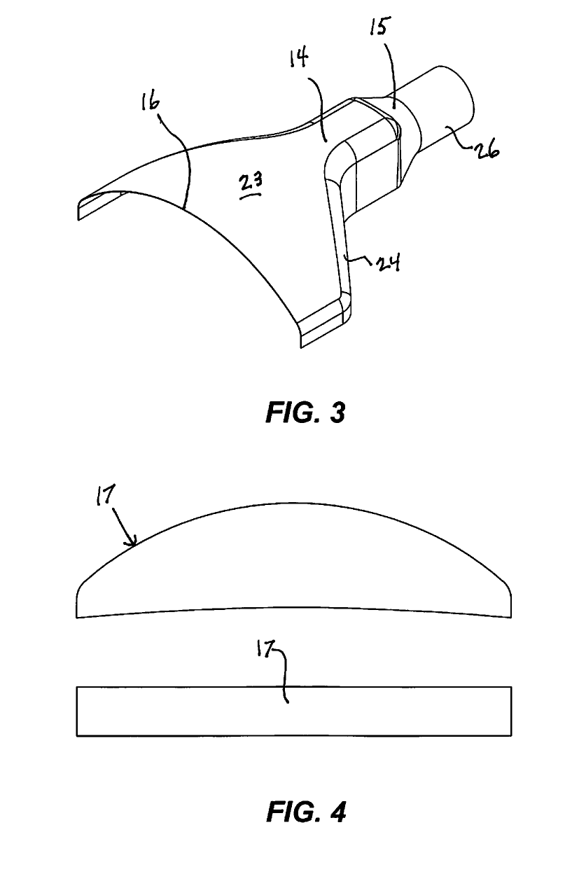

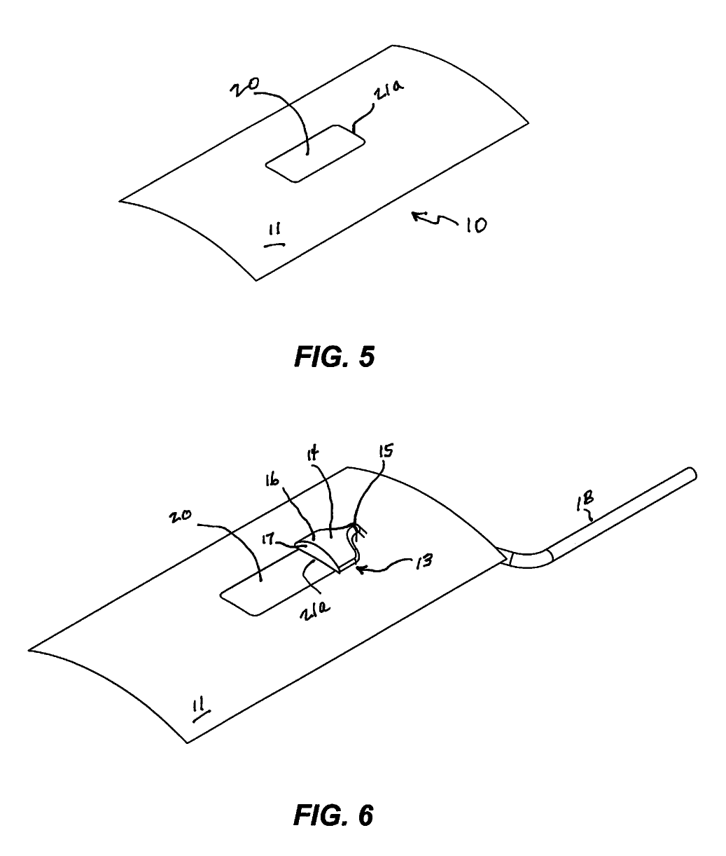

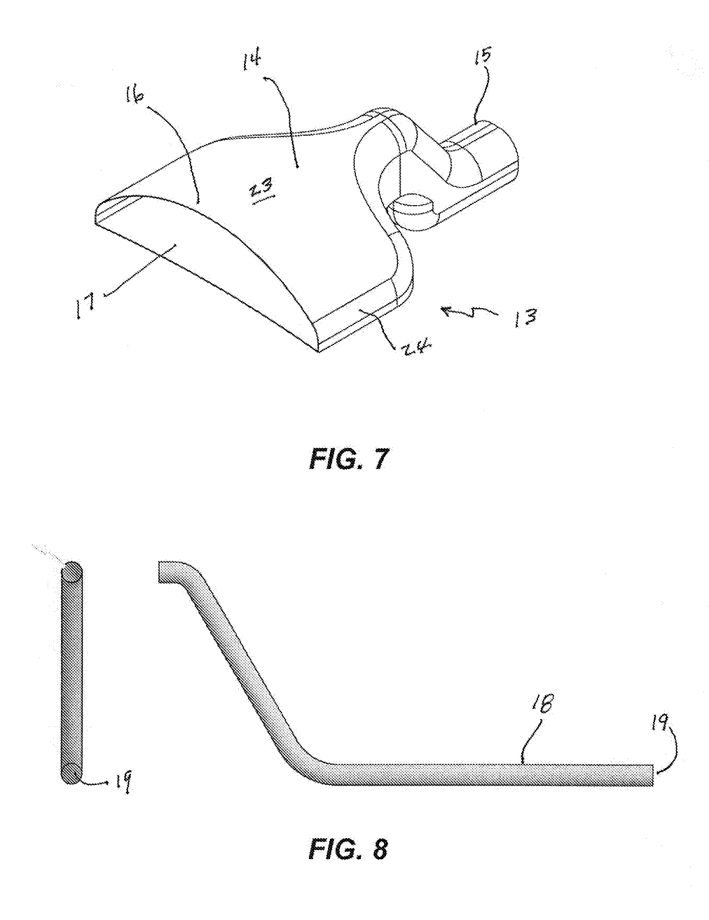

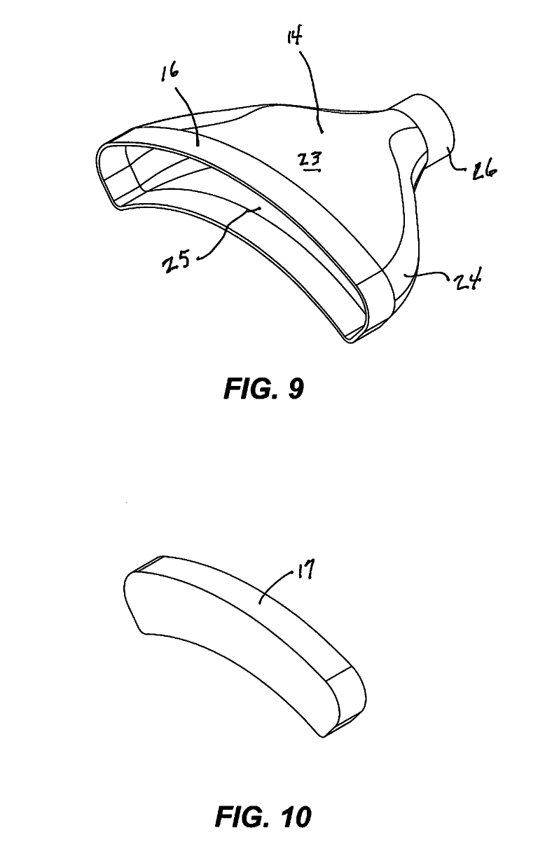

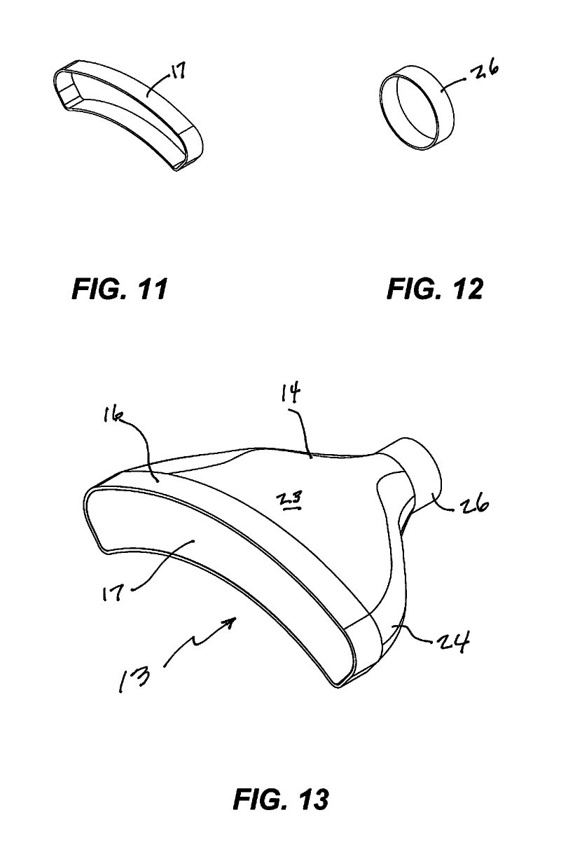

Referring first to FIGS. 1-8, a surgical drape generally indicated by the reference numeral 10 comprises a topside 11, a bottom side 12, and an emitter generally indicated by reference numeral 13 sealed to a portion of the drape as more particularly described below. Referring also to FIGS. 9-15 in addition to FIGS. 1-8, the emitter 13 comprises an inflatable receiver portion 14 for receipt of a flow of sterile gas through an inlet 15 and a terminal outlet portion 16 fluidly continuous with receiver portion 14. The terminal outlet portion 16 contains a frontally planar gas distributor 17, suitably an open cell foam, for example, a reticulated open cell flexible ether type of polyurethane foam (e.g., FIGS. 4, 10, 13, 15). A gas conduit 18 is joined at least in part to drape 10 for receipt of sterile gas flow from a source of sterile gas and for connection to the inlet 15 of the receiver portion 14 of the emitter 13. The gas conduit 18 may have a bottom or top side bonded respectively to the topside or bottom side of the drape and be inflatable on receipt of sterile gas flow from a source of sterile gas. Such a structure may have folded lateral sides that unfold on receipt of sterile air to inflate the gas conduit. Alternatively, at least a portion of topside 11 of drape 10 extending from emitter 13 distally to a gas conduit inlet 19 for connection to a source of sterile gas is pneumatically nonporous, and gas conduit 18 has a top and lateral sides and no bottom surface and is sealed at the edges of its lateral sides to the pneumatically nonporous portion of topside 11. Still alternatively, as depicted in FIG. 8, gas conduit 18 may comprise a hose 18 for receipt of sterile gas flow from a source of sterile gas and for connection to inlet 15 of the receiver portion 14 of the emitter 13 but not be joined to drape 10. In FIGS. 1, 2, gas conduit 18 is partially on topside 11. In FIGS. 5-8, the gas conduit 18 rises from the bottom side 12 of drape 10 passing through an aperture in drape 10 for connection to inlet 15 of the receiver portion 14 of the emitter 13.

In the embodiments of FIGS. 1-15, distributor 17 fills the terminal outlet portion 16 of emitter 13. As seen in FIGS. 1, 2, 5 and 6 surgical drape 10 includes an opening 20 extending completely through drape 10. In the embodiment, opening 20 is a quadrilateral bounded by bordering sides 21a-21d and drape 10 is for attachment to a portion of a patient that is not an extremity. As seen in FIG. 2, the bottom side 12 of drape 10 has an adhesive 22 surrounding opening 20, and such an adhesive is implicit for drape 10 of FIGS. 5, 6. Adhesive 22 provides releasable adherence of drape 10 about a site of the surface anatomy of a patient that is not an extremity where a surgical incision is to be made. The terminal outlet portion 17 of emitter 13 is close to one of the sides of the opening, as depicted, side 21a and is frontally as wide as bordering side 21a.

Two types of emitters are depicted. In FIGS. 1, 3, 4 6 and 7, emitter 13 has top or roof 23 and lateral sides 24 and no bottom surface. The emitter of FIGS. 1, 3, 4, 6 and 7 is employed with a surgical drape 10 having an opening 20 and in which at least a portion of the topside 11 of drape 10 close to opening 20 is pneumatically nonporous. The emitter in FIGS. 1, 3, 4 6 and 7 is sealed at the bottom edges of the lateral sides 24 to the pneumatically nonporous portion of topside 11. In FIG. 10, distributor 17 is illustrated removed from emitter 13. With distributor 17 removed, emitter 13 is seen in FIG. 9 to have a bottom surface 25. Bottom surface 25 will be bonded to the topside 11 of drape 10 close to one of the sides of opening 16, e.g., side 21a, and as in the depictions of the emitter 13 of FIGS. 1 and 6 having no bottom surface. In the emitter of FIGS. 9-15 the topside 11 of drape 10 close to opening 20 does not need to be pneumatically nonporous. In the emitter 13 of FIGS. 9-15, the terminal outlet portion 16 of emitter 13 may be a separate flexible plastic material such as silicone into which the distributor 17 may be fixedly sealingly fitted. The receiver portion 14 suitably is an inflatable flexible plastic film such as a vinyl film. In such an embodiment, the forepart of the inflatable material of the receiving portion 14 of the emitter is suitably sealed to the outside of the terminal outlet portion 16, as in FIGS. 9, 13 and 15. In an alternative embodiment, the forepart of the terminal outlet portion may also be a continuation of the same material as forms the receiving portion 14 and is suitably sealed directly to the perimeter of distributor 17. In the embodiment of FIGS. 9-15, the rear part of the inflatable material of the receiving portion 14 of the emitter is suitably sealed to the outside of a ring 26 for connection to a gas conduit 18. Ring 26 may be a flexible material such as silicone.

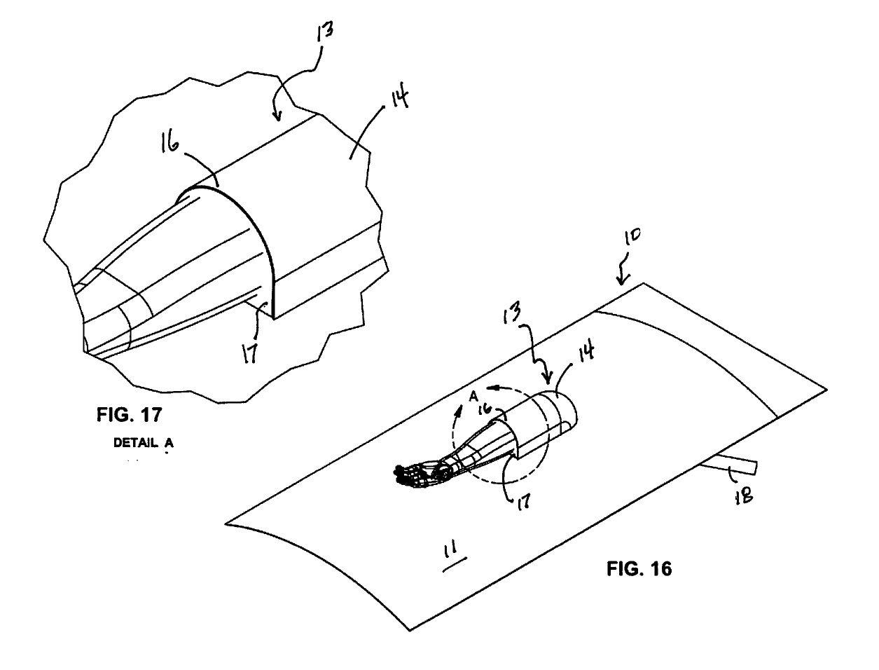

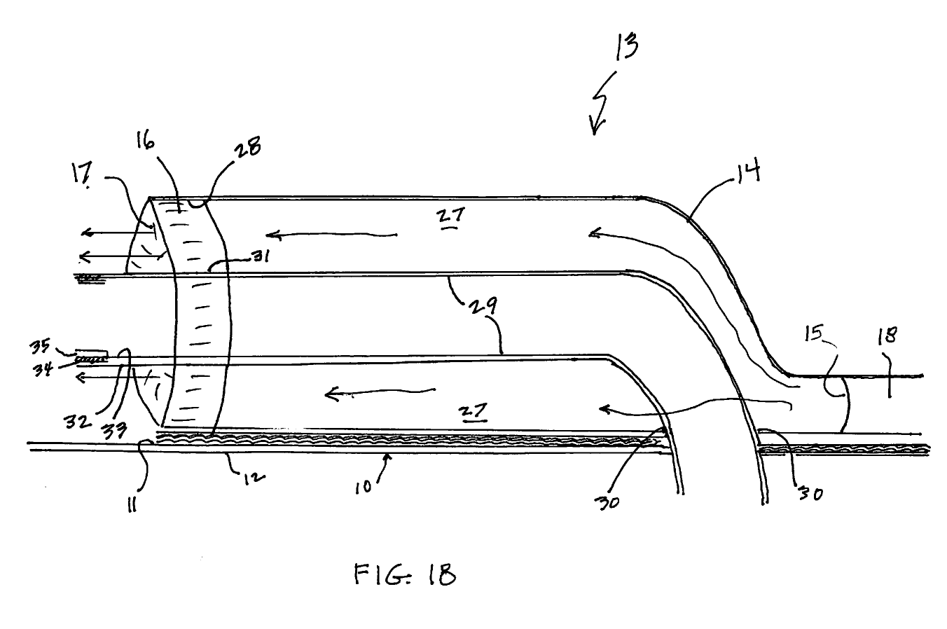

Another embodiment of a surgical drape 10 is depicted in FIGS. 16, 17 and 18, which depict the drape 10 with inflatable receiver portion inflated. This embodiment of a surgical drape is for a patient who is having surgery on an extremity. Referring to FIGS. 16-18, surgical drape 10 has a topside 11 and a bottom side 12, has an emitter 13 that connects to a gas conduit 18. Emitter 13 comprising an inflatable receiver portion 14 having an inlet 15 for sealingly connection to gas conduit 18 and a terminal outlet portion 16 fluidly continuous with receiver portion 14. Receiver portion 14 and terminal outlet portion 17 comprise an annulus 27. Terminal outlet portion 16 contains a frontally planar annular gas distributor 17 within annulus 27. Annular distributor 17 is externally bonded to an interior 28 of annulus 27. Drape 10 further includes a sleeve 29 externally bonded to an interior 30 of receiver portion 14 distal from annular distributor 17 and is externally bonded nonporously to an interior circumference 31 of annular distributor 17. Sleeve 29 accepts an extremity of a patient with a portion of the extremity extending past annular distributor 17. Sleeve 29 ends in a cuff 32 immediately past annulus 27. Cuff 32 has an interior 33 that includes an adhesive 34 covered by a removable strip 35. Conditioned sterile gas flow emitted from the terminal outlet portion coherently proceeds levelly to a site of incision generally perpendicular to a radius of the extremity.

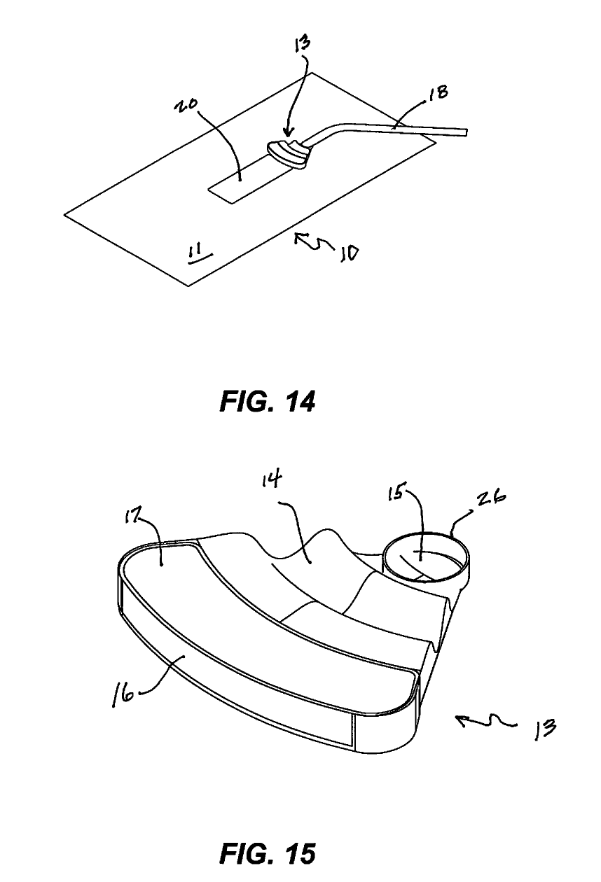

Surgical drape 10 is foldable essentially flat with the emitter collapsed within the drape, suitably within folds of the drape. As depicted in FIGS. 14 and 15 with drape 10, unfolded to reveal the interior, as collapsed the receiver portion 14 is deflated and the terminal outlet portion 16 containing distributor 17 is folded over into a prone position essentially parallel to the drape. In an embodiment in which the gas conduit 18 is also inflatable, drape 10 is foldable essentially flat with the emitter 13 and gas conduit 18 collapsed within the drape, suitably within folds of the drape.

Surgical drape 10 may be provided folded in a surgical procedure kit for a surgical site that is not an extremity of a patient. Such a kit in an embodiment could comprise a folded essentially flat surgical drape 10 containing a deflated inflatable gas conduit 18 joined to drape 10 and an emitter 13, drape 10 having a topside 11 and a bottom side 12, at least a portion of the topside 11 being pneumatically nonporous, emitter 13 being hermetically sealed to at least a portion of the pneumatically nonporous topside 11 of drape 10, emitter 13 comprising a deflated inflatable receiver portion 14 having an inlet 15 sealingly connected to gas conduit 18 and a terminal outlet portion 16 fluidly continuous with receiver portion 14, the terminal outlet portion 16 containing a frontally planar gas distributor 17.

In one such surgical kit, distributor 17 fills the terminal outlet portion 16 and terminal outlet portion 16 and distributor 17 are prone in the kit, and drape 10 further comprises an opening 20 extending completely through the drape, opening 10 being quadrilateral and bounded by bordering sides 21a-d, bottom side 12 of the drape having an adhesive 22 surrounding opening 20 for releasable adherence of drape 10 about a site that is not an extremity of the surface anatomy of a patient where a surgical incision is to be made, the pneumatically nonporous portion of topside 12 being close to one of the bordering sides of the opening, and the terminal outlet portion 16 of the emitter being frontally as wide as a bordering side of the opening 20 where the portion of the drape topside 11 is pneumatically nonporous, in consequence of which upon pneumatic inflation of the receiver portion 14, terminal outlet portion 16 erects and conditioned flow emitted from the terminal outlet portion 16 proceeds levelly over opening 11 from only one bordering side of opening 11.

A method of protecting a patient from surgical site infection from an incision in the anatomy of the patient that is not an extremity during surgery in an operating room where the site is exposed to operating room atmosphere making use of such a surgical kit would comprises (a) providing the surgical procedure kit, (b) unfolding the drape and arranging the drape over the patient with the opening placed over a site of the surface anatomy of a patient that is not an extremity where a surgical incision is to be made, such placement locating the flow conditioning terminal outlet portion of the emitter at one side of the site of the surgical incision, (c) adhering the drape to the patient at the sides of the opening thereby anatomically shape conforming the flow conditioning terminal outlet portion of the emitter to the anatomical surface of a patient, (d) connecting the gas conduit to a source of sterile gas, and (e) supplying a flow of sterile gas to the gas conduit and the emitter to pneumatically inflate the gas conduit and the receiver portion of the emitter and erect the terminal outlet portion and distributor to emit a conditioned unidirectional coherent non-turbulent flow field of essentially sterile gas into ambient air in the operating room substantially anatomically levelly over the anatomical surface of the patient within the opening from one side of the site of incision to an opposite side thereof for a distance at least immediately past the incision while keeping the gas essentially sterile, the sealing of the emitter inclusive of the distributor to the pneumatically nonporous topside of the drape and the adherence of the drape to the patient preventing entry of ambient air under the emitter into the emitted flow.

In another surgical kit in which a drape 10 is provided folded for surgery at a surgical site that is an extremity of a patient, the kit comprises a folded essentially flat surgical drape 10 containing a deflated inflatable gas conduit 18 joined to drape 10 and an emitter 13, drape 10 having a topside 11 and a bottom side 12, at least a portion of the topside 11 being pneumatically nonporous, emitter 13 being hermetically sealed to at least a portion of the pneumatically nonporous topside 11 of drape 10, emitter 13 comprising a deflated inflatable receiver portion 14 having an inlet 15 sealingly connected to gas conduit 18 and a terminal outlet portion 16 fluidly continuous with receiver portion 14, the terminal outlet portion 16 containing a frontally planar gas distributor 17. The receiver portion 14 and terminal outlet portion 16 comprise an annulus 27 and distributor 17 within annulus 27 is annular and externally bonded to an interior 28 of annulus 27. Drape 10 further includes a sleeve 29 externally bonded to an interior 30 of receiver portion 14 distal from annular distributor 27 and is externally bonded nonporously to an interior circumference 31 of annular distributor 17. Sleeve 29 accepts an extremity of a patient with a portion of the extremity extending past annular distributor 17, sleeve 29 ending in a cuff 32 immediately past annulus 27, cuff 32 having an interior 33 that includes an adhesive 34 covered by a removable strip 35.

A method of protecting a patient from surgical site infection from an incision in the anatomy of the patient that is an extremity during surgery in an operating room where the site is exposed to operating room atmosphere, making use of such a surgical kit comprises (a) providing the surgical procedure kit, (b) unfolding the drape and inserting the extremity of the patient into the sleeve with a portion of the extremity where an incision is to be made extending through the distributor past the cuff, such placement locating said flow conditioning terminal outlet portion annulus at one side of the site of the surgical incision, (c) removing said strip from the adhesive and adhering the cuff to the extremity at the cuff thereby anatomically shape conforming said flow conditioning terminal outlet portion annulus to the anatomical surface of a patient where an incision is to be made, (d) connecting the gas conduit to a source of sterile gas, and (e) supplying a flow of sterile gas to the gas conduit and the emitter to pneumatically inflate the gas conduit and the receiver portion of the emitter to emit from the annular distributor a conditioned unidirectional coherent non-turbulent flow field of essentially sterile gas into ambient air in the operating room substantially anatomically levelly generally perpendicular to a radius of the extremity a distance at least immediately past the incision while keeping the gas essentially sterile, said sealing of the emitter inclusive of said distributor to said pneumatically nonporous topside of the drape and said adherence of the drape cuff to the patient preventing entry of ambient air under said emitter into said emitted flow.

The above disclosed subject matter is to be considered illustrative, and not restrictive, and the appended claims are intended to cover all modifications, enhancements, and other embodiments that fall within the true scope of the present invention, which to the maximum extent allowed by law, is to be determined by the broadest permissible interpretation of the following claims and their equivalents, unrestricted or limited by the foregoing detailed descriptions of exemplary embodiments of the invention

1. A surgical drape comprising a topside, a bottom side, and an emitter sealed to a portion of the drape and comprising an inflatable receiver portion for receipt of a flow of sterile gas and a terminal outlet portion fluidly continuous with said receiver portion, said terminal outlet portion containing a frontally planar gas distributor, said receiver portion pneumatically inflating upon receipt of a sterile gas flow producing backpressure at said distributor, said distributor of said terminal outlet portion upon inflation of the receiver portion conditioning said received sterile gas flow into a coherent non-turbulent flow emitted unidirectionally from said terminal outlet portion.

2. The surgical drape of claim 1 in which said emitter has a bottom side sealed to the topside of the drape.

3. The surgical drape of claim 1 in which at least a portion of said topside is pneumatically nonporous, the emitter has a top and lateral sides and no bottom side and the emitter is sealed at the edges of said lateral sides to said pneumatically nonporous portion of said topside.

4. The surgical drape of claim 1 further comprising a gas conduit joined to the drape for receipt of sterile gas flow from a source of sterile gas and connection to said receiver portion of said emitter.

5. The surgical drape of claim 4 in which said gas conduit has a bottom or top side bonded respectively to the topside or bottom side of the drape, and is inflatable on receipt of sterile gas flow from a source of sterile gas.

6. The surgical drape of claim 4 in which at least a portion of said topside extending from said emitter distally to an inlet for connection to a source of sterile gas is pneumatically nonporous, said gas conduit has a top and lateral sides and no bottom side and is sealed at the edges of said lateral sides to said pneumatically nonporous portion of said topside.

7. The surgical drape of claim 1 in which said drape is combined with a gas conduit comprising a hose not joined to said drape, for connection to said receiver portion of said emitter on said topside of said drape.

8. The surgical drape of claim 1 in which said distributor fills the terminal outlet portion of said emitter.

9. The surgical drape of claim 8 in which the terminal outlet material is a different material than an inflatable material of said receiver portion and a forepart of the material of said receiver portion is sealed to the outside of the terminal outlet portion.

10. The surgical drape of claim 8 in which the terminal outlet portion is a continuation of material the same as the material of which the receiver portion is formed and in which the continuation material is sealingly bonded to the perimeter of the distributor.

11. The surgical drape of claim 8 further comprising an opening extending completely through the drape, said opening being bounded by bordering sides, said bottom side having an adhesive surrounding said opening for releasable adherence of the drape about a site of the surface anatomy of a patient where a surgical incision is to be made.

12. The surgical drape of claim 11 in which said opening is quadrilateral.

13. The surgical drape of claim 12 in which said terminal outlet portion of said emitter is close to one of said boundary sides of said opening.

14. The surgical drape of claim 13 in which said terminal outlet portion of said emitter is frontally as wide as a bordering side of said opening to which the terminal outlet portion of the emitter is close.

15. The surgical drape of claim 11 in which at least a portion of said topside is pneumatically nonporous, the emitter has lateral sides and no bottom side, the emitter is sealed at the edges of said lateral sides to said pneumatically nonporous portion of said topside, and said pneumatically nonporous portion of said topside is close to one of the boundary sides of said opening.

16. The surgical drape of claim 11 in which said opening is for attachment to a portion of a patient that is not an extremity and said flow emitted from said terminal outlet portion proceeds levelly over said opening from only one bordering side of said opening.

17. The surgical drape of claim 1 in which said receiver portion and said terminal outlet portion is an annulus and said distributor within said annulus is annular and externally bonded to an interior of said annulus, for surroundingly accepting an extremity of a patient though said distributor.

18. The surgical drape of claim 17 in which said drape includes a sleeve externally bonded to an interior of said collapsible inflatable receiver portion distal from said annular distributor and is externally bonded nonporously to an interior circumference of said annular distributor, said sleeve accepting said extremity with a portion of said extremity extending through and past said distributor.

19. The surgical drape of claim 18 in which sleeve ends in a cuff immediately past said annulus and said cuff has an interior that includes an adhesive covered by a removable strip.

20. The surgical drape of claim 19 in said flow emitted from said terminal outlet portion proceeds to a site of incision coherently generally perpendicular to a radius of the extremity.

21. The surgical drape of claim 1 in which said drape is foldable essentially flat with said emitter collapsed within folds of said drape.

22. The surgical drape of claim 21 in which said drape is foldable essentially flat also with said gas conduit collapsed within folds of said drape.

23. The surgical drape of claim 21 folded with said terminal outlet portion of said emitter containing said distributor facing said drape and with said receiver portion deflated behind said terminal outlet portion.

24. The surgical drape of claim 1 in which said receiver portion of said emitter comprises a roof of larger inflated area than an area of the topside of the drape under said roof.

25. The surgical drape of claim 1 in which said emitter is formed of a flexible plastic.

26. The surgical drape of claim 25 in which said emitter is formed of a flexible plastic film.

27. A surgical procedure kit comprising a folded essentially flat surgical drape containing a deflated inflatable gas conduit joined to the drape and an emitter, said drape having a topside and a bottom side, at least a portion of said topside being pneumatically nonporous, said emitter being hermetically sealed to at least a portion of said pneumatically nonporous topside of the drape, said emitter comprising a deflated inflatable receiver portion having an inlet sealingly connected to said gas conduit and a terminal outlet portion fluidly continuous with said receiver portion, said terminal outlet portion containing a frontally planar gas distributor, said receiver portion pneumatically inflating upon receipt of sterile gas flow from said gas conduit producing backpressure at said distributor, said distributor of said terminal outlet portion upon inflation of the receiver portion conditioning said received sterile gas flow into a coherent non-turbulent flow emitted unidirectionally from said terminal outlet portion.

28. The surgical procedure kit of claim 27 in which said distributor fills said terminal outlet portion of the emitter, said drape further comprising an opening extending completely through the drape, said opening being bounded by bordering sides, said bottom side having an adhesive surrounding said opening for releasable adherence of the drape about a site of the surface anatomy of a patient that is not an extremity where a surgical incision is to be made, said pneumatically nonporous portion of said topside being close to one of said bordering sides of said opening.

29. The surgical procedure kit of claim 28 in which opening is quadrilateral, said terminal outlet portion of said emitter is frontally as wide as a bordering side of said opening where said portion of said drape topside is pneumatically nonporous, and in which said flow emitted from said terminal outlet portion proceeds levelly over said opening from only one bordering side of said opening.

30. The surgical kit of claim 29 in which said terminal outlet portion is prone when said receiver portion is deflated and in which pneumatic inflation of said receiver portion erects said terminal outlet portion.

31. The surgical procedure kit of claim 27 in which said receiver portion and said terminal outlet portion comprise an annulus and said distributor within said annulus is annular and externally bonded to an interior of said annulus, and in which said drape includes a sleeve externally bonded to an interior of said receiver portion distal from said annular distributor and is externally bonded nonporously to an interior circumference of said annular distributor, said sleeve accepting said extremity with a portion of said extremity extending past said distributor, said sleeve ending in a cuff immediately past said annulus, said cuff having an interior that includes an adhesive covered by a removable strip.

32. A method of protecting a patient from surgical site infection from an incision in the anatomy of the patient that is not an extremity during surgery in an operating room where the site is exposed to operating room atmosphere, comprising
providing a surgical procedure kit comprising a folded essentially flat surgical drape having a topside, a bottom side, and an opening extending completely through the drape, said opening being bounded by bordering sides, said bottom side having an adhesive surrounding said opening, said drape a deflatable inflatable gas conduit joined to the drape and an emitter, at least a portion of said topside close to one of said sides of said opening being pneumatically nonporous, said emitter being hermetically sealed to at least a portion of said pneumatically nonporous topside of the drape, said emitter comprising a deflated inflatable receiver portion having an inlet sealingly connected to said gas conduit and a prone terminal outlet portion fluidly continuous with said receiver portion, said terminal outlet portion containing a frontally planar gas distributor filling said terminal outlet portion, said gas conduit pneumatically inflating upon receipt of sterile gas flow from a source of sterile gas and said receiver portion pneumatically inflating upon receipt of sterile gas flow from said gas conduit producing backpressure at said distributor and erecting said terminal outlet portion, said distributor of said terminal outlet portion upon inflation of the emitter conditioning said received sterile gas flow into an emitted coherent non-turbulent flow,
unfolding said drape and arranging the drape over the patient with said opening placed over a site of the surface anatomy of a patient that is not an extremity where a surgical incision is to be made, such placement locating said flow conditioning terminal outlet portion of said emitter at one side of the site of the surgical incision,
adhering the drape to the patient at the sides of the opening thereby anatomically shape conforming said flow conditioning terminal outlet portion of the emitter to the anatomical surface of a patient,
connecting said gas conduit to a source of sterile gas,
supplying a flow of sterile gas to said gas conduit and said emitter to pneumatically inflate said gas conduit and said receiver portion of said emitter and erect said terminal outlet portion to emit from said distributor a conditioned unidirectional coherent non-turbulent flow field of essentially sterile gas into ambient air in the operating room substantially anatomically levelly over the anatomical surface of the patient within said opening from one side of the site of incision to an opposite side thereof for a distance at least immediately past the incision while keeping the gas essentially sterile, said sealing of the emitter inclusive of said distributor to said pneumatically nonporous topside of the drape and said adherence of the drape to the patient preventing entry of ambient air under said emitter into said emitted flow.

33. A method of protecting a patient from surgical site infection from an incision in the anatomy of the patient that is an extremity during surgery in an operating room where the site is exposed to operating room atmosphere, comprising
providing a surgical procedure kit comprising a folded essentially flat surgical drape having a topside and a bottom side, at least a portion of said topside being pneumatically nonporous, said drape containing a deflated inflatable gas conduit joined to the drape and an emitter, said emitter being hermetically sealed to at least a portion of said pneumatically nonporous topside of the drape, said emitter comprising a deflated inflatable receiver portion having an inlet sealingly connected to said gas conduit and a terminal outlet portion fluidly continuous with said receiver portion, said receiver portion and said terminal outlet portion comprising an annulus, said terminal outlet portion annulus containing an annular frontally planar gas distributor externally bonded to an interior of said annulus, said drape including a sleeve externally bonded to an interior of said inflatable receiver portion of said emitter distal from said terminal outlet portion annulus and externally bonded nonporously to an interior circumference of said annular distributor, said sleeve ending in a cuff immediately past said terminal outlet portion annulus, said cuff having an interior that includes an adhesive covered by a removable strip, said sleeve accepting an extremity of a patient with a portion of said extremity extending through and past said annular distributor, said gas conduit pneumatically inflating upon receipt of sterile gas flow from a source of sterile air, said receiver portion pneumatically inflating upon receipt of sterile gas flow from said gas conduit producing backpressure at said annular distributor, and said annular distributor conditioning said received sterile gas flow into an emitted coherent non-turbulent flow from said terminal outlet portion annulus,
unfolding said drape and inserting the extremity of the patient into the sleeve with a portion of the extremity where an incision is to be made extending through the distributor past the cuff, such placement locating said flow conditioning terminal outlet portion annulus at one side of the site of the surgical incision,
removing said strip from the adhesive and adhering the cuff to the extremity at the cuff thereby anatomically shape conforming said flow conditioning terminal outlet portion annulus to the anatomical surface of a patient where an incision is to be made,
connecting said gas conduit to a source of sterile gas flow,
supplying a flow of sterile gas to said gas conduit and said emitter to pneumatically inflate said gas conduit and said receiver portion of said emitter to emit from said distributor a conditioned unidirectional coherent non-turbulent flow field of essentially sterile gas into ambient air in the operating room substantially anatomically levelly generally perpendicular to a radius of the extremity a distance at least immediately past the incision while keeping the gas essentially sterile, said sealing of the emitter inclusive of said distributor to said pneumatically nonporous topside of the drape and said adherence of the drape cuff to the patient preventing entry of ambient air under said emitter into said emitted flow.
